(19) United States
(12) Patent Application Publication
(10) Pub. No.: US 2013/0053661 A1
(43) Pub. Date: Feb. 28, 2013

(54) SYSTEM FOR ENABLING RELIABLE SKIN CONTRACT OF AN ELECTRICAL WEARABLE DEVICE

(71) Applicants: William P. Alberth, Prairie Grove, IL (US); Rachid M. Alameh, Crystal Lake, IL (US)

(72) Inventors: William P. Alberth, Prairie Grove, IL (US); Rachid M. Alameh, Crystal Lake, IL (US)

(73) Assignee: MOTOROLA MOBILITY, INC., Libertyville, IL (US)

(21) Appl. No.: 13/222,293

(22) Filed: Aug. 31, 2011

(51) Int. Cl. A61B 5/04; A61B 5/1455

(52) U.S. Cl. 600/322; 600/390

(57) ABSTRACT

A system for enabling reliable skin contact of an electrical wearable device associated with physiological information detected by the wearable device. The system includes a wearable tightness detector powered by a power supply for the wearable device, and a controller for monitoring electrical signals emanating from the power supply and the wearable tightness detector. Also, at least two thermocouples electrically coupled to the wearable tightness detector, along with at least two skin contact modules electrically coupled to the wearable tightness detector. The at least two thermocouples provide the physiological information detected by the wearable device in relation to a subject. An adjustable wearable band is included in the system. The tightness of the wearable band is detected by the wearable tightness detector and an output signal from the at least two thermocouples to ensure reliable physiological information from the subject during the subject's wearing of the wearable device.

### FIELD OF THE DISCLOSURE

The present disclosure relates generally to electrical wearable devices and more particularly to electrical wearable devices capable of providing physiological information about the subject wearing the electrical wearable device.

### BACKGROUND

Some electrical wearable devices have incorporated galvanic skin response measurement contacts into their systems. A galvanic skin response detector measures a voltage difference across two sensors that are placed in contact with the skin. If the connection is not consistent then the measurement will not be accurate.

Accordingly, there is a need for a system for enabling reliable skin contact of an electrical wearable device.

### BRIEF DESCRIPTION OF THE FIGURES

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 is an illustrative example of a system comprising an electrical wearable device attached to an adjustable band.

FIG. 2 is an illustrative example of circuitry useful in detecting tightness of the adjustable band shown in FIG. 1.

FIG. 3 is an exemplary block diagram of optional physiological sensors that may be employed by the electrical wearable device.

FIG. 4 is an illustrative schematic of an exemplary antenna configuration for the electrical wearable device.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The system and apparatus components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

A system is disclosed herein for enabling reliable skin contact of an electrical wearable device and prevention of false signals associated with physiological information detected by the wearable device. The system includes a wearable tightness detector powered by a power supply for the wearable device; and controlled by a controller for monitoring electrical signals emanating from the power supply and the wearable tightness detector. In addition, at least two thermocouples electrically can be coupled to the wearable tightness detector, along with at least two skin contact modules electrically coupled to the wearable tightness detector. The at least two thermocouples provide the physiological information detected by the wearable device in relation to a subject. An adjustable wearable band is included in the system. The tightness of the wearable band is detected by the wearable tightness detector and an output signal from the at least two thermocouples to ensure reliable physiological information from the subject during the subject's wearing of the wearable device.

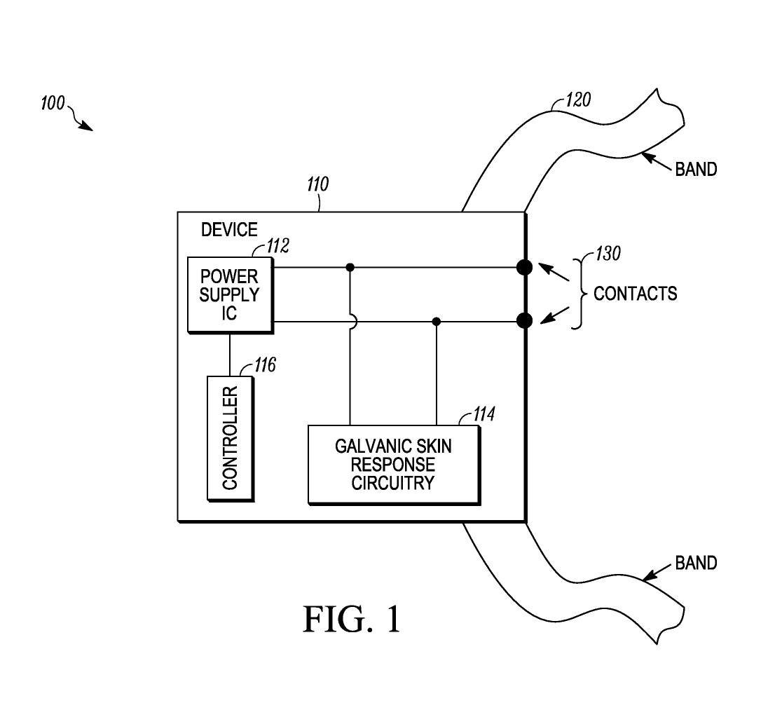

FIG. 1 is an illustrative example of a system 100 comprising an electrical wearable device 110 attached to an adjustable band 120. The wearable device 110 may include at least two protruding or flat contacts 130 for sensing external data corresponding to health or physiology of a subject wearing the wearable device 110. These contacts are preferably on or integrated with the electrical wearable device 110, but could also be integrated within the adjustable band 120. Contacts 130 may also serve an independent function of being a means for charging the wearable device 110. Circuitry associated with the wearable device 110 can include a power supply 112 that may be a separate component or may reside on an integrated chip; a wearable tightness detector 114; and a controller 116 for monitoring and adjusting electrical signals emanating from the power supply 112 and the wearable tightness detector 114. The wearable tightness detector 114 may also serve to provide a Galvanic Skin Response measurement. Since the wearable tightness detector 114 essentially measures the voltage difference between two contacts, it can also be used to measure the voltage output from two thermocouples.

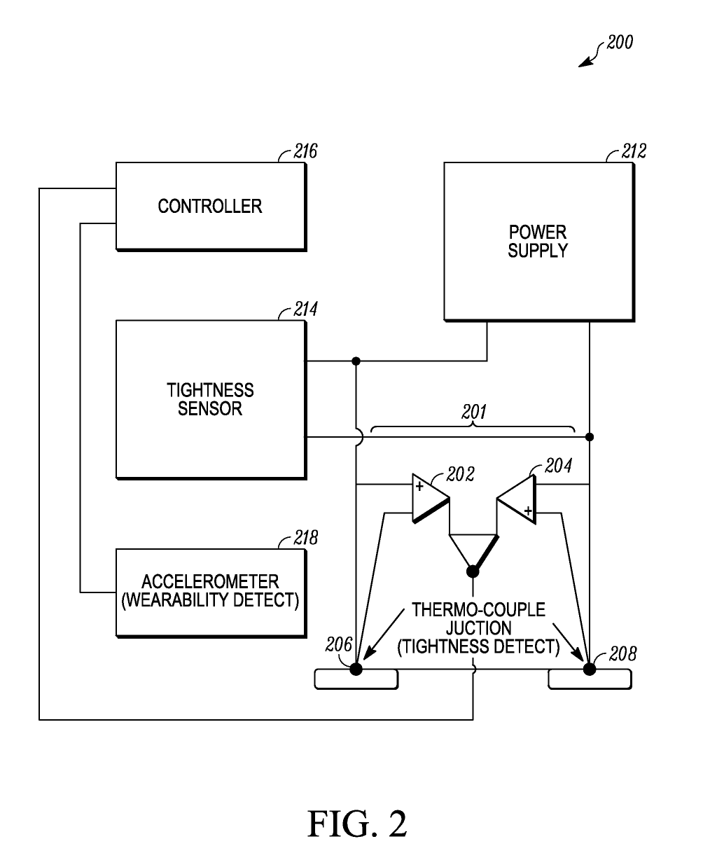

The adjustable band 120 or in many cases the electrical wearable device 110 can also be monitored for relative tightness against a subject's skin during wearing of the wearable device. FIG. 2 is an illustrative example of circuitry 200 useful in detecting tightness of the adjustable band 120 shown in FIG. 1. Circuitry 200 includes a thermo-couple circuit 201 comprised of amplifiers 202 and 204 for controlling input signals from sensors 206 and 208. The output 209 indicates temperature variance between the thermo couples 206 and 208. The thermo-couple circuit 201 is electrically and communicatively coupled to a wearable tightness detector 214 and power supply 212. Accelerometer circuitry 218 is electrically coupled to a controller 216. Accelerometer circuitry 218 detects movement of the wearable device and can be considered as a wearability detection that emits output signals that change with time.

Controller 216 of circuit 200 can manage and control signals associated with the accelerometer circuit 218, the tightness detector 214, and the power supply 212. Specific to tightness measurement, the tightness measured output is detected when thermo-couple sensors 206 and 208 are exposed to similar temperatures (for example, output signal is substantially zero). When the wearable device is loose on a wrist of a subject, the thermo-couple sensors 206 and 208 will not sense the exact same skin temperature because some portion of the adjustable band is contacting the skin, while another portion of the adjustable band is exposed to ambient air. If the band is not tight, one of the thermocouples will be touching the skin, while the other thermocouple does not contact the subject's skin, therefore, the individual thermocouples 206 and 208 will be at a different temperature from each other. Tightness detector 214 detects this differential. Accordingly, the output signal of the thermo-couple circuit 201 can either be positive or negative. In other words, both thermo-couple sensors 206 and 208 should detect substantially the same temperature emanating from the skin of the subject; otherwise, the adjustable band cannot be considered tight against the skin of the subject. By monitoring the tightness of the adjustable band, reliable contact against the skin of the subject can be ensured. Upon determining that reliable contact exists, other sensors such as a heart rate sensor can be employed with greater assurance of true responses.

The system 100 may also include a heart rate monitor, for example. Heart rate monitors require constant pressure against the skin to enable reliable readings. When the controller 216 determines that the band is tight then the controller 216 can activate the heart rate monitor.

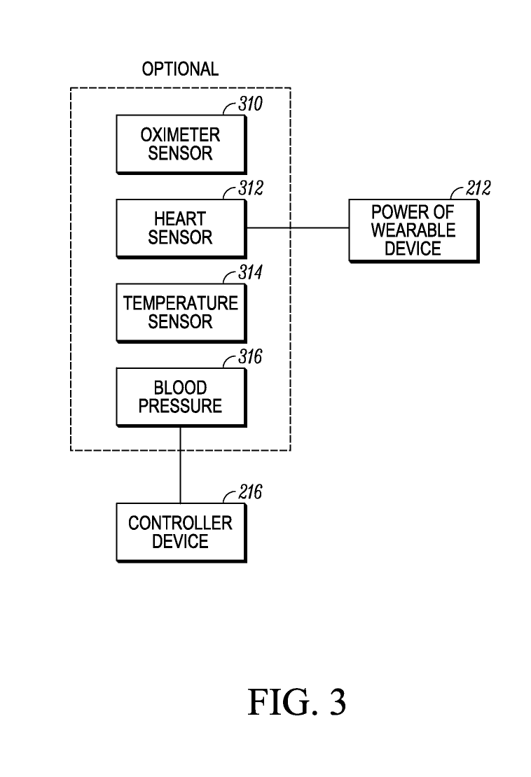

FIG. 3 is an exemplary block diagram of optional biometric sensors 300 that may be employed by the electrical wearable device and that are operation when the adjustable wearable band is detected to have sufficient contact to the subject's skin. Optional biometric sensors 300 are electrically and communicatively coupled to controller 216 and the power supply 212 (shown in FIG. 2). Optional biometric sensors 300 can include a pulse oximeter sensor 310 for measuring or detecting oxygen saturation of a subject's blood. Optional biometric sensors 300 can include a heart rate sensor 312 for measuring or detecting a subject's heart rate. The heart rate sensor may be an optical transceiver type (not shown) for reflecting light off a subject's blood hemoglobin. The optical transceiver may be designed to reflect off the blood vessels or penetrate through the blood vessels. This could be achieved by locating the optical transmitter and receiver on the same side of a body part belonging to the subject wearing the electrical wearable device or locate the optical transmitter and corresponding receiver on opposite sides of the subject's body part, respectively. Optional biometric sensors 300 can include a temperature sensor 314 for measuring or detecting a subject's body temperature. Optional biometric sensors 300 can include a blood pressure sensor 316 for measuring or detecting a subject's blood pressure. These optional biometric sensors are not contemplated as exhaustive and therefore, can including other physiological sensing technology as well, for example, a perspiration sensor, a skin conductivity sensor, or a melanoma sensor.

Accelerometer 218 (shown in FIG. 2) can be employed to evaluate accuracy of sensor output based on the wearable device's motion. Additionally, duty cycle and/or measurement times of the biometric sensors can be adjusted to conserve power relative to the wearable device's motion, its static orientation, its dynamic acceleration, its detected vibration readings, or operational mode of the electrical wearable device. For example, if the wearable device receives accelerometer measurements indicative of the subject or user running, then a heart rate monitor's duty cycle could be increased. If the accelerometer output is indicative of the subject being at rest, then the heart rate monitor duty cycle could be decreased. The accelerometer may also provide signals that enable electrical power to the sensor to be enabled or disabled based on the wearable device's motion as detected via the accelerometer.

The electrical wearable device's context can be assessed and evaluated, via output sensors, to provide greater accuracy to and about the subject. For example, one can determine whether the electrical wearable device is in a dark or light environment, whether the wearable device is in motion or at rest, or whether the electrical wearable device is in a noisy or quiet environment.

The electrical wearable device can include a telecommunication antenna apparatus for transmitting and receiving radio frequency signals when the electrical wearable device's connectors/contacts are not in contact with the user. The telecommunication antenna apparatus can be, for example, a dual inverted L-antenna. Alternatively, one or more skin contacts of the wearable device can be converted into a low power telecommunication antenna when the skin contact(s) are touching the subject's skin. When the electrical wearable device's connectors are in contact with the subject's body, the subject's body can be used as part of the antenna with well known tuning apparatus. Notably, the human body can be deemed to have a resonance near the broadcast spectrum of about 100 MHz or less.

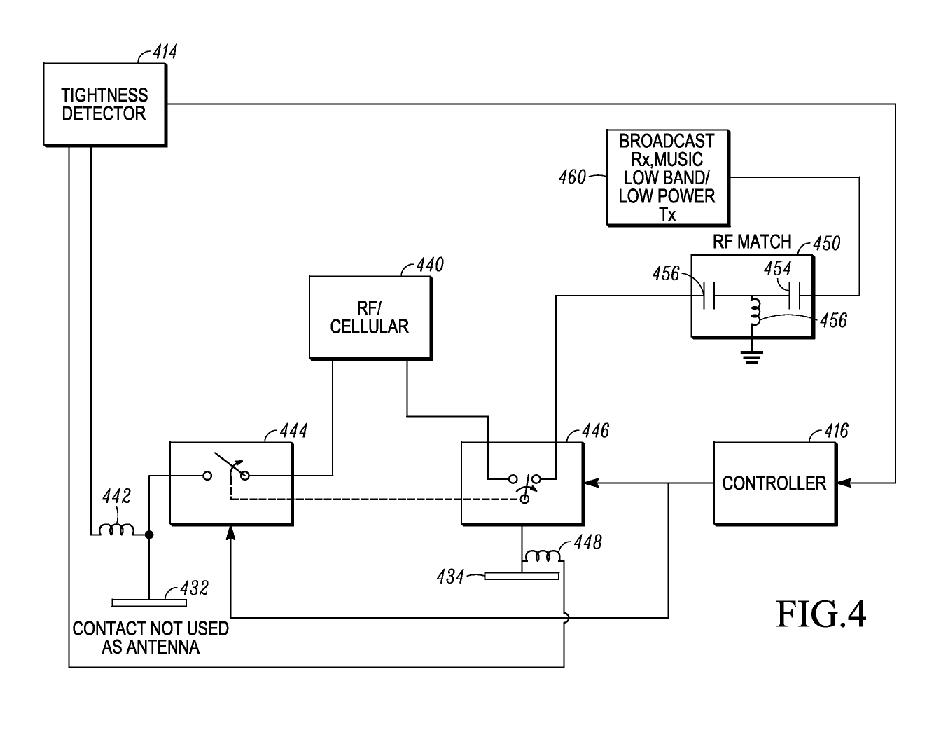

FIG. 4 is an illustrative schematic of an exemplary antenna configuration 400 for the electrical wearable device. A radio frequency (RF) or cellular antenna 440 may be electrically coupled to skin contact 432 and 434 via switches 444 and 446. Skin contact 434 is electrically coupled to a RF choke element/coil 448 to form an antenna. Whereas, skin contact 432 that is electrically coupled to RF choke coil 442 and switch 444 does not act as an antenna in this exemplary embodiment. These types of antennas are of non intrinsic ground types. Other antenna types that utilize both connectors can be implemented as well; including, for example, a low power antenna (useful for example as a broadcast receiver).

A RF impedance match 450 is generally comprised of at least a coil 452 and two capacitors 454, 456, respectively and is used to tune to channels associated with the low power transceiver 460. The switch 446 selects between the cellular transceiver and the low power broadcast transceiver 460. Transceiver 460 can comprise a receiver and/or a transmitter. A tightness detector 414 electrically coupled to the antenna configuration 400 along with electrically coupled controller 416 enable the transmission and reception of broadcast signals depending on the tightness of the adjustable wearable band against the subject's skin as detected by the skin contacts that are adhered to the adjustable wearable band. The skin contacts can be glued or electrically integrated (via trace conductivity or electrical coating, for example) into the adjustable wearable band as well as sewn into the band, or the device's bottom side (where the device protrudes through the band and contacts the skin).

In addition to implementation of a telecommunication antenna apparatus, the electrical wearable device can include skin contact modules that also provide connection to a voltage from a power supply (as shown in FIGS. 1 and 2) for the electrical wearable device. A voltage reading associated with the power supply can be taken at the skin contact modules, thereby to provide a voltage reading of the power supply. The skin contact modules can monitor whether any sensed voltage reading crosses a predetermined charging threshold for the electrical wearable device. For example, the predetermined charging threshold may be at least 2 volts. The electrical wearable device, therefore, can begin charging from the power supply when the charging threshold is exceeded. The contacts 432 and 434 may be connected to two leads to a power supply (not shown in FIG. 4) to charge the electrical wearable device. Since the wearable tightness detector 414 is measuring the voltage different from the contacts, it can also be used to detect the voltage from the charging power supply.

If the voltage across 432 and 434 exceeds a threshold, for example, 2V as determined by the tightness detector, then the electrical wearable device 110 can determine that a charger has been attached to the contacts 432 and 434 and the voltage can be applied to a circuit to recharge a battery or power supply (not shown) in system 100. If the voltage falls below a threshold, say 1.8V, then the wireless device 110 (as shown in FIG. 1) can determine that the charger has been disconnected.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way, but may also be configured in ways that are not listed.

It will be appreciated that some embodiments may be comprised of one or more generic or specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the method and/or apparatus described herein. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used.

Moreover, an embodiment can be implemented as a computer-readable storage medium having computer readable code stored thereon for programming a computer (e.g., comprising a processor) to perform a method as described and claimed herein. Examples of such computer-readable storage mediums include, but are not limited to, a hard disk, a CD-ROM, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

We claim:

1. A system for enabling reliable skin contact of an electrical wearable device and prevention of false signals associated with physiological information detected by the wearable device, comprising:
a wearable tightness detector;
a power supply for the electrical wearable device;
a controller for monitoring electrical signals emanating from the power supply and the wearable tightness detector;
at least two thermocouples electrically coupled to the wearable tightness detector;
at least two skin contact modules electrically coupled to the wearable tightness detector and the at least two thermocouples for providing the physiological information detected by the wearable device in relation to a subject; and
an adjustable wearable band, wherein tightness of the wearable band is detected by the wearable tightness detector and an output signal from the at least two thermocouples to ensure reliable physiological information from the subject during the subject's wearing of the electrical wearable device.

2. The system according to claim 1, further comprising a sensor related to biometric readings of the subject, wherein the biometric readings are selected from the group consisting of the subject's heart rate, blood pressure, skin conductivity, oxygen, and temperature.

3. The system according to claim 2, wherein a heart rate sensor comprises an optical transceiver for reflecting light off blood hemoglobin of the subject.

4. The system according to claim 3, wherein the heart rate sensor comprises an optical transceiver for receiving light after propagating through a body part.

5. The system according to claim 2, wherein the sensor related to biometric readings of the subject is operational when the adjustable wearable band is detected to have sufficient contact to the subject's skin.

6. The system according to claim 2, wherein outputs from the sensor are evaluated for accuracy based on the wearable device's motion as detected via an accelerometer.

7. The system according to claim 2, wherein duty cycle and/or measurement times of the biometric sensor is adjusted relative to either the wearable device's motion, its static orientation, its dynamic acceleration, its detected vibration readings, or its operational mode.

8. The system according to claim 4, wherein an output from the wearable tightness detector is an indication of sufficient contact to a subject's skin.

9. The system according to claim 1, wherein the wearable band is detachable from the wearable device and the at least two skin contact modules, and includes a telecommunication antenna apparatus for transmitting and receiving radio frequency signals when the wearable band is not in contact with the subject.

10. The system according to claim 1, wherein the at least two skin contacts of the wearable device are converted into a telecommunication antenna when the at least two skin contacts are touching the subject's skin.

11. The system according to claim 1, wherein the at least two skin contact modules provide a voltage reading of the power supply.

12. The system according to claim 1, wherein the voltage reading from the at least two skin contact modules crosses a predetermined charging threshold.

13. The system according to claim 1, wherein the predetermined charging threshold is at least 2 volts.

14. The system according to claim 12, wherein the electrical wearable device charges from the power supply when the charging threshold is exceeded.

15. The system according to claim 12, wherein the wearable tightness detector is deactivated during charging.

16. The system according to claim 1, wherein the at least two skin contact modules are in the adjustable wearable band.

17. The system according to claim 1, wherein the at least two skin contact modules are on the bottom side of the electrical wearable device and protrude through the adjustable wearable band for contacting skin of the subject.

18. The system according to claim 2, wherein outputs from the sensor are evaluated for accuracy based on the wearable device's context.

19. The system according to claim 2, wherein power to the sensor is enabled or disabled based on the wearable device's motion as detected via an accelerometer.
